(19) United States
(12) Patent Application Publication
(10) Pub. No.: US 2025/0356482 A1
(43) Pub. Date: Nov. 20, 2025

(54) NON-VISIBLE-SPECTRUM LIGHT IMAGE-BASED OPERATIONS FOR VISIBLE-SPECTRUM IMAGES

(71) Applicant: Intuitive Surgical Operations, Inc., Sunnyvale, CA (US)

(72) Inventors: Anthony M. Jarc, Johns Creek, GA (US); Theodore W. Rogers, Alameda, CA (US)

(21) Appl. No.: 18/874,382

(22) PCT Filed: Jun. 14, 2023
(86) PCT No.: PCT/US2023/025292

§ 371 Date: Dec. 12, 2024

Related U.S. Application Data
(60) Provisional application No. 63/352,839, filed on Jun. 16, 2022.

(51) Int. Cl. G06T 7/00; H04N 23/50; H04N 23/56

(52) U.S. Cl. CPC G06T 7/0012; H04N 23/555; H04N 23/56; G06T 2207/10016; G06T 2207/10048; G06T 2207/10064

(57) ABSTRACT

An illustrative system may access a first image sequence comprising first images, the first images based on illumination, using visible-spectrum light, of a scene associated with a medical procedure; access a second image sequence comprising second images, the second images based on illumination of the scene using non-visible spectrum light; detect an object in the second image sequence; and perform, based on the detected object, an operation with respect to the first image sequence.

### RELATED APPLICATIONS

The present application claims priority to U.S. Provisional Patent Application No. 63/352,839, filed Jun. 16, 2022, the contents of which is hereby incorporated by reference in its entirety.

### BACKGROUND INFORMATION

Light-based image data captured during medical procedures has many uses, during such procedures and after. For example, medical image data from an endoscope can be displayed during a medical procedure to help medical personnel carry out the procedure. As another example, medical image data captured during a medical procedure can be used as a control signal for computer-assisted medical systems. As another example, medical image data captured during a medical procedure may also be used after the medical procedure for post-procedure evaluation, diagnosis, instruction, and so forth.

A variety of illuminating and image-sensing technologies have been used to capture images of medical procedures. Visible-spectrum illuminants and image sensors have been used to capture color (white light) images of medical procedures. Non-visible-spectrum image sensors, sometimes paired with non-visible-spectrum illuminants, have been used to capture non-visible-spectrum images of medical procedures.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An illustrative system includes a memory storing instructions; and one or more processors communicatively coupled to the memory and configured to execute the instructions to perform a process comprising: accessing a first image sequence comprising first images, the first images based on illumination, using visible-spectrum light, of a scene associated with a medical procedure; accessing a second image sequence comprising second images, the second images based on illumination of the scene using non-visible spectrum light; detecting an object in the second image sequence; and performing, based on the detected object, an operation with respect to the first image sequence.

An illustrative method performed by one or more computing devices may include: accessing a first image sequence comprising first images, the first images based on illumination, using visible-spectrum light, of a scene associated with a medical procedure; accessing a second image sequence comprising second images, the second images based on illumination of the scene using non-visible spectrum light; detecting an object in the second image sequence; and performing, based on the detected object, an operation with respect to the first image sequence.

An illustrative non-transitory computer-readable medium may store instructions that, when executed, direct a processor of a computing device to perform a process comprising: accessing a first image sequence comprising first images, the first images based on illumination, using visible-spectrum light, of a scene associated with a medical procedure; accessing a second image sequence comprising second images, the second images based on illumination of the surgical scene using non-visible spectrum light; detecting an object in the second image sequence; and performing, based on the detected object, an operation with respect to the first image sequence.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows a system for capturing images of a scene associated with a medical procedure.

FIG. 2 shows an image processing system generating outputs that may be used to perform operations with respect to a first image sequence.

FIG. 3 shows an embodiment where more than two image sequences are captured and used.

FIG. 4 shows a technique for capturing a scene in first and second image sequences.

FIG. 5 shows a technique for generating synthetic second images.

FIG. 6 shows an embodiment for labeling a first image sequence based on detecting and/or recognizing objects in a second image sequence.

FIG. 7 shows an embodiment for segmenting a first image sequence.

FIG. 8 shows an embodiment for modifying a first image sequence.

FIG. 9 shows an embodiment for generating and using a three-dimensional (3D) model.

FIG. 10 shows a computer-assisted medical system.

FIG. 11 shows an illustrative computing device.

### DETAILED DESCRIPTION

Imaging of a scene associated with a medical procedure has many applications. For example, the display of images of an obscured scene associated with a medical procedure may allow a surgeon performing the medical procedure to see and evaluate the obscured scene or may allow a surgeon to see displayed information not otherwise available from direct observation. Different types of images have been used to capture and portray scenes associated with medical procedures. For example images of visible light have been used, as have images of non-visible light. Previously, these different types of images have been used independently. For example, during a surgical procedure, the different image types may be displayed alternatively, one then the other. The information in two or more different types of images may not be leveraged at the same time. For example, although two types of images of a scene might be available, only the information in one might be used or displayed at any given time. Information that could be used to improve or augment the other image may go unused.

Techniques for using non-visible-spectrum images to enable operations performed with respect to visible-spectrum images are described herein. Given a first sequence of images (e.g., visible-spectrum images) of a scene and a second sequence of images (e.g., non-visible-spectrum images) of the scene, object detection and/or recognition may be performed on the second sequence and outputs of the object detection and/or recognition may be used to perform operations with respect to the first image sequence. For example, based on object detection performed on the second sequence, a label may be applied to the first image sequence, which may assist a surgeon or other user in performing a surgical procedure while viewing the first image sequence. This and other advantages and benefits of using non-visible-spectrum images to enable operations performed with respect to visible-spectrum images are described in detail herein.

As used herein, “visible-spectrum image” and “visible-spectrum video” refer to images and video whose pixel values represent sensed intensities of visible-spectrum light. “Non-visible-spectrum image” and “non-visible-spectrum video” refer to images and video whose pixel values represent sensed intensities of non-visible-spectrum light. For brevity, “image” will be used herein to refer to both images and video. Illustrative non-visible-spectrum images include fluorescence images, hyperspectral images, and other types of images that do not rely solely on visible-spectrum illumination. For example, fluorescence images are images of light fluoresced from matter when the matter is illuminated by a non-visible-spectrum illuminant. Infrared images are another type of non-visible-spectrum image. Infrared images are images captured by sensors that can sense light in an infrared wave range. For example, the infrared light may include light emitted by illuminated fluorophores.

As used herein, a “label” refers to any type of data indicative of an object or other feature represented in an image including, but not limited to, graphical or text-based annotations, tags, highlights, augmentations, and overlays. A label applied to an image may be embedded as metadata in an image file or may be stored in a separate data structure that is linked to the image file. A label can be presented to a user, for example, as an augmentation to the image, or may be utilized for other purposes that do not necessarily involve presentation such as training of a machine learning model.

As used herein, a “medical procedure” can refer to any procedure in which manual and/or instrumental techniques are used on a patient to investigate, diagnose, or treat a physical condition of the patient. Additionally, a medical procedure may refer to any non-clinical procedure, e.g., a procedure that is not performed on a live patient, such as a calibration or testing procedure, a training procedure, and an experimental or research procedure.

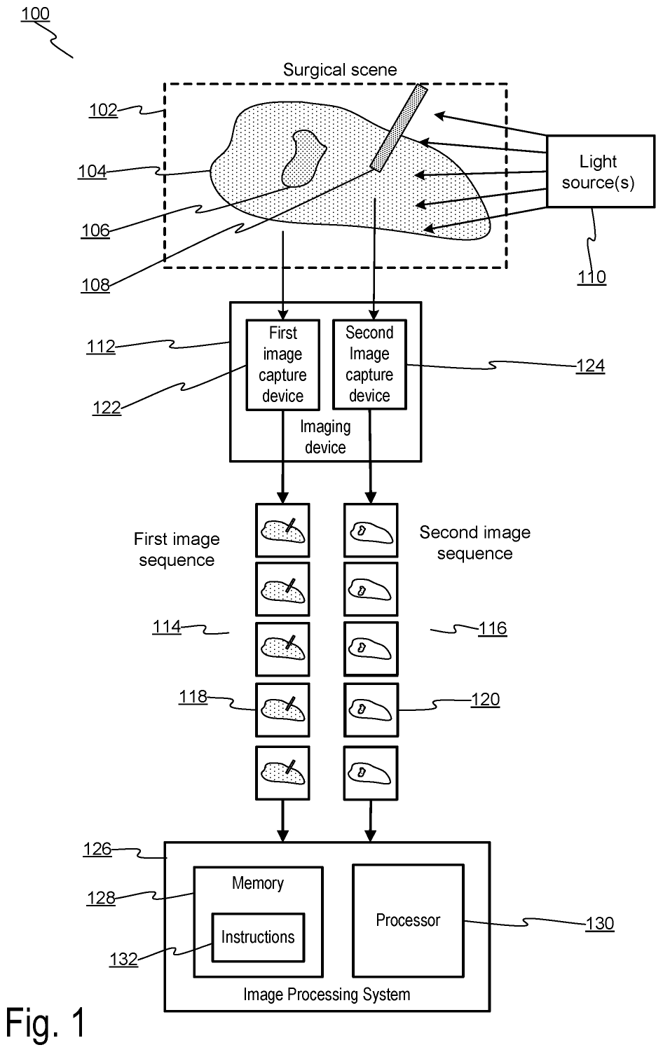

FIG. 1 shows a system 100 implementing a method for capturing images of a scene 102 associated with a medical procedure. The scene 102 may include a surgical area associated with a body on or within which the medical procedure is being performed (e.g., a body of a live animal, a human or animal cadaver, a portion of human or animal anatomy, tissue removed from human or animal anatomies, non-tissue work pieces, physical training models, etc.). For example, the scene 102 may include various types of tissue (e.g., tissue 104), organs (e.g., organ 106), and/or non-tissue objects (e.g., object 108) such as instruments, objects held or manipulated by instruments, etc.

One or more light sources 110 may illuminate the scene 102. As noted above, the light sources 110 might include any combination of a white light source, a narrow-band light source (whether in the visible spectrum or not, e.g., an ultraviolet lamp), a laser, an infrared light emitting diode (LED), etc. If fluoresced light is to be captured, the type of light source may depend on the fluorescing agent or protein being used during the medical procedure. In some implementations, a light source might provide light in the visible spectrum but the fluoresced light that it induces may be out of the visible spectrum.

Further regarding fluoresced light, in some implementations of the system 100, a light source for fluorescence illumination (i.e., an excitation light source) may have any wavelength outside the visible spectrum. For example, a fluorescence illuminant, such as indocyanine green (ICG), may produce light with a wavelength in an infrared radiation region (e.g., about 700 nm to 1 mm), such as a near-infrared (“NIR”) radiation region (e.g., about 700 nm to 950 nm), a short-wavelength infrared (“SWIR”) radiation region (e.g., about 1,400 nm to 3,000 nm), or a long-wavelength infrared (“LWIR”) radiation region (e.g., about 8,000 nm to 15,000 nm). In some examples, a fluorescence illuminant may produce light in a wavelength of approximately 1000 nm or greater (e.g., SWIR and LWIR). Additionally, or alternatively, the fluorescence illuminant may output light with a wavelength of about 350 nm or less (e.g., ultraviolet radiation). In some implementations, the fluorescence illuminant may be specifically configured for optical coherence tomography imaging.

The system 100 also includes an imaging device 112. The imaging device 112 receives light reflected, emitted, and/or fluoresced from the subject of the medical procedure and converts the received light to image data. The imaging device 112 senses light from the scene 102 and outputs a first image sequence 114 and a second image sequence 116 of the scene 102. The first image sequence 114 may be a sequence of visible-spectrum images 118 of light sensed in the visible spectrum. The second image sequence 116 may be a sequence of non-visible-spectrum images 120 of light sensed in a non-visible-spectrum. The image sequences may be in the form of individual images, an encoded video stream, etc. As shown in FIG. 1, because the image sequences are from different spectrums (or partially non-overlapping spectrums), the content of the respective image sequences may differ; some features of the scene may be represented in one sequence and not the other.

The imaging device 112 may have a first image capture device 122 and a second image capture device 124. Either image capture device may be any type of device capable of converting photons to an electrical signal, for example a charge-coupled device (CCD), a complementary metal oxide semiconductor (CMOS) sensor, a photo multiplier, etc. Regardless of the type of image capture devices used, the imaging device 112 may be configured to sense light in both the visible spectrum and outside the visible spectrum, as noted above. In some embodiments, the first image capture device 122 senses light in the visible spectrum, and the second image capture device 124 senses light in a non-visible spectrum. The first image capture device 122 and the second image capture device 124 may be separate sensors within a single camera, or they may be separate sensors in separate respective cameras. In some embodiments, the imaging device 112 may include only one image capture device (e.g., one sensor), and the image capture device is capable of concurrently sensing in the visible spectrum and in one or more non-visible spectrums. For example, some image sensors are capable of simultaneously sensing in the visible spectrum and in an infrared spectrum. In other embodiments, the imaging device 112 may be a stereoscopic camera and may have two cameras each capable of sensing in the visible spectrum and a non-visible spectrum. In some embodiments, the imaging device 112 and the light sources 110 may be part of (or optically connected with) an endoscope.

In one embodiment, the first image capture device 122 may continuously capture the first image sequence 114 as video data of the medical procedure, and the second image capture device 124 may capture the images of the second image sequence 116 intermittently. For example, the first image capture device 122 might capture a video frame (first image 118) every 60th of a second and the second image capture device 124 might capture a second image 120 once every second.

As shown in FIG. 1, the image processing system 126 may be configured to access (e.g., receive) the first image sequence 114 and the second image sequence 116 to perform various operations with respect to the first image sequence 114, as described below.

The image processing system 126 may be implemented by one or more computing devices and/or computer resources (e.g., processors, memory devices, storage devices, etc.) as may serve a particular implementation. As shown, the image processing system 126 may include, without limitation, a memory 128 and a processor 130 selectively and communicatively coupled to one another. The memory 128 and the processor 130 may each include or be implemented by computer hardware that is configured to store and/or process computer software. Various other components of computer hardware and/or software not explicitly shown in FIG. 1 may also be included within the image processing system 126. In some examples, the memory 128 and the processor 130 may be distributed between multiple devices and/or multiple locations as may serve a particular implementation.

The memory 128 may store and/or otherwise maintain executable data used by the processor 130 to perform any of the functionality described herein. For example, the memory 128 may store instructions 132 that may be executed by the processor 130. The memory 128 may be implemented by one or more memory or storage devices, including any memory or storage devices described herein, that are configured to store data in a transitory or non-transitory manner. The instructions 132 may be executed by the processor 130 to cause the image processing system 126 to perform any of the functionality described herein. The instructions 132 may be implemented by any suitable application, software, code, and/or other executable data instance. Additionally, the memory 128 may also maintain any other data accessed, managed, used, and/or transmitted by the processor 130 in a particular implementation.

The processor 130 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), digital signal processors, or the like. Using the processor 130 (e.g., when the processor 130 is directed to perform operations represented by the instructions 132 stored in the memory 128), the image processing system 126 may perform various operations as described herein.

Various implementations of the image processing system 126 will now be described with reference to the figures and how the image processing system 126 may be configured to implement modules described below. The various modules described herein may be included in the image processing system 126 and may be implemented by any suitable combination of hardware and/or software. As such, the modules represent various functions that may be performed by the image processing system 126 alone or in combination with any of the other functions described herein as being performed by the image processing system 126 and/or a component thereof.

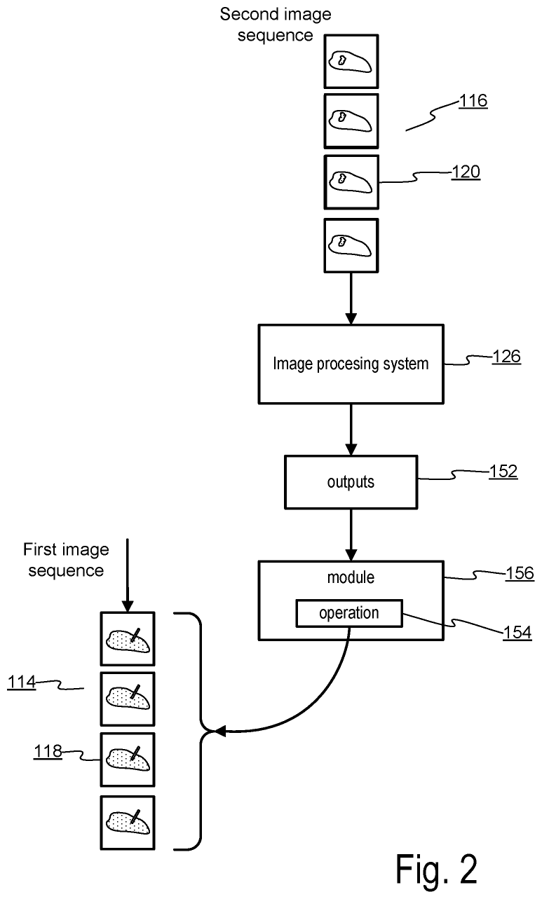

FIG. 2 shows the image processing system 126 generating outputs 152 that may be used to perform operations 154 with respect to the first image sequence 114. The image processing system 126 receives the second image sequence 116. The image processing system 126 may be configured to perform various image processing algorithms on the second image sequence 116. For example, the image processing system 126 may implement any known object detection and/or recognition algorithms to detect and/or recognize objects in the second image sequence 116.

In some embodiments, the outputs 152 may include indications of objects detected or recognized in the second image sequence 116. For example, an output 152 may be a region or blob corresponding to a region of contiguous pixels having values within a given color range, a detected perimeter (e.g., a set of detected edges), an object detected based on inter-frame movement detection, a segment derived from a segmentation algorithm (e.g., foreground-background segmentation) implemented by the image processing system 126, a region isolated by a time-varying signal (e.g., “pulsing” pixel values), and so forth. In some embodiments, the outputs 152 may include indications of objects recognized in the second image sequence 116. For example, the outputs 152 may be labels or tags of recognized types or categories of objects and may include information linking the labels to their respective graphic portions in corresponding first images (i.e., the labels may be linked to locations in the first images). In some embodiments, labels or tags may be associated with individual images but not particular objects therein.

The outputs 152 from the image processing system 126 may be provided to a module 156 that performs an operation 154 with respect to the first image sequence 114 based on outputs 152. The operation 154 may include, for example, labeling one or more first images 118, augmenting one or more first images 118 (e.g., by brightening, highlighting, adding text to, etc.), labeling one or more objects in a first image 118 that correspond to labeled objects recognized in the second image sequence 116, etc. The operation 154 may include, for example, segmenting one or more first images 118 according to the outputs 152. The outputs 152 may be segments that can be directly applied or mapped to the first images 118, or they may be detected objects that provide an additional input for finding segments in the first images 118 using an augmented segmentation algorithm. In other embodiments, the outputs 152 are used to modify or transform the image content of one or more of the first images 118. For example, a region in a first image 118 might be enhanced (e.g., brightened) based on a corresponding output 152. An object detected and/or recognized in the second image sequence 116 may be overlaid or blended into a first image 118. A graphic text label may be added to a first image 118. These operations 154 and others are described below.

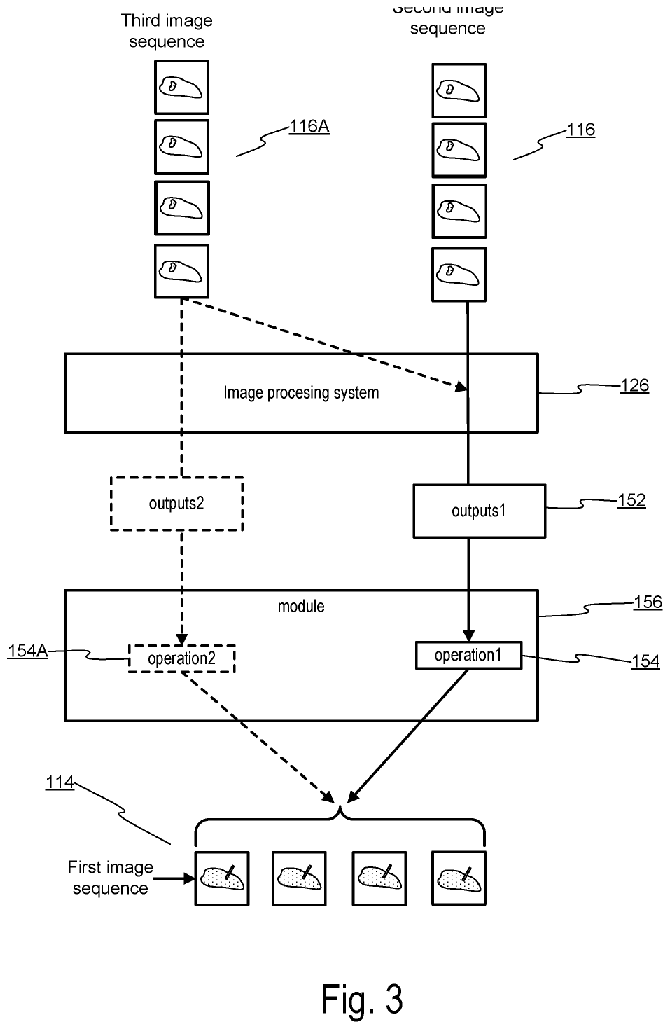

FIG. 3 shows an embodiment where more than two image sequences are captured and used to perform various operations described herein. For example, the first image sequence 114 may be visible-spectrum images, the second image sequence 116 may be fluorescence images, and a third image sequence 116A may correspond to another imaging modality such as hyperspectral imaging. In such embodiments, the second and/or third image sequences 116/116A may be captured in the background (e.g., while the first image sequence 114 is being captured) or by using sampling techniques described later. Sampling for the second and third image sequences 116, 116A may be at a different rate or at the same rate but offset. Further, the second and third images sequences 116, 116A can be processed together or independently to perform operations with respect to the first image sequence 114. For example, when processing independently, the second image sequence 116 may be processed to perform a first operation 154 (e.g., detecting a first type of anatomical feature) and the third image sequence 116A may be processed to perform a second operation 154A (e.g., detecting a second type of anatomical feature). When processed together, the second and third image sequences 116, 116A may both contribute to output 152 provided to the first operation 154, e.g., to improve object detection.

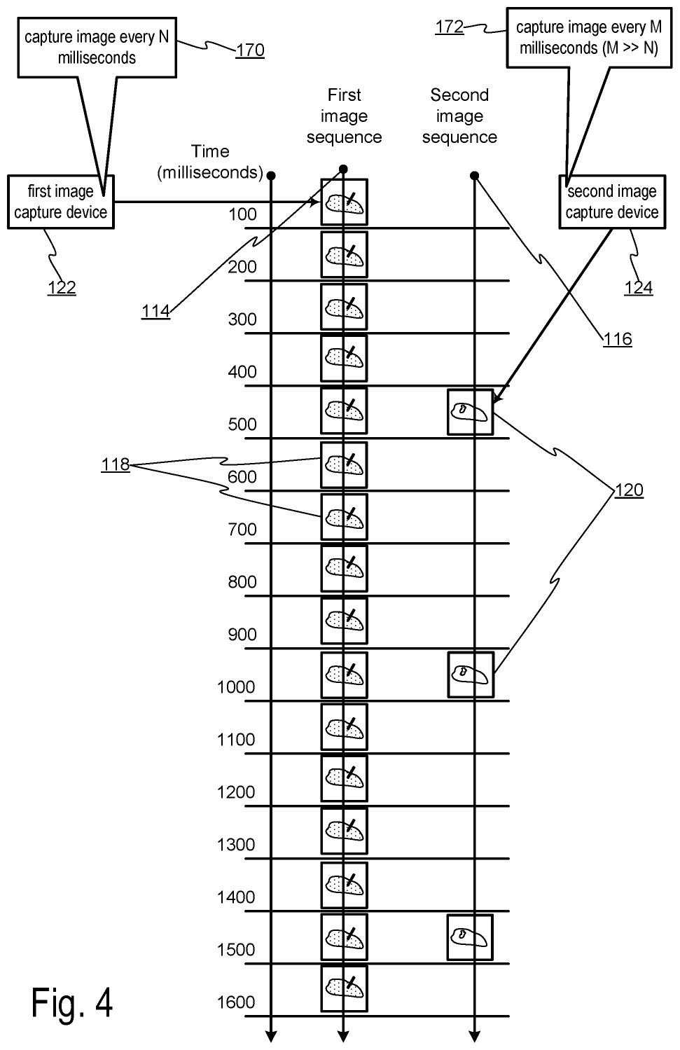

FIG. 4 shows a technique for capturing a scene 102 in the first and second image sequences 114, 116. As discussed above, it is not uncommon to use two imaging techniques or modes during a surgical procedure. Some imaging techniques may be challenging to use concurrently. For example, if a particular type of illuminating light is needed for one imaging mode, that type of light might be interfered with by (or interfere with) the other imaging mode. In some imaging systems, concurrently using two imaging modes might present problems such as heat accumulation due to the added illumination energy, increased data, etc. In addition, in some imaging systems, there may be other factors that may limit the ability to concurrently capture images or video in two different modes. For example, some medical imaging systems (e.g., endoscopes) may have two capture modes and two respective display modes, and the modes may operate in a mutually exclusive manner so that image or video data for one mode can only be captured when that mode is being actively displayed. Furthermore, some imaging modes may require a long sensing phase (e.g., 1 second) and may not be capable of sensing new images at a faster rate (e.g., 1 per second).

FIG. 4 shows a technique for capturing the first and second image sequences 114, 116 in a way that may avoid some challenges associated with concurrently capturing in two different types of imaging modes. As discussed above, the first image capture device 122 captures the first image sequence 114. The first image capture device 122 may perform a first operation 170 of capturing a first image 118 every N milliseconds. N may be small enough for the first image sequence 114 to be rendered as video. For example, the first image capture device 122 may capture a first image 118 every 16.7 milliseconds (i.e., every 1/60th of a second). A high frame/image capture rate for the first image capture device 122 may be sufficient to render the first image sequence 114 as video on a display, i.e., a live view of the scene 102 may be displayed. For explanation, in the example shown in FIG. 4, the first image capture device 122 captures a first image 118 every 100 milliseconds.

The second image capture device 124 performs a second operation 172 of capturing a second image 120 every M milliseconds, where M is less than N. In some embodiments, N is substantially more than M. For example, M may be twice N. That is, the second image capture device 124 may capture images at half the rate of the first image capture device 122. In one embodiment, N may be 16.7 milliseconds and M may be 1000 milliseconds, i.e., one second image 120 is captured for every 60 first images 118 that are captured. In the example shown in FIG. 4, M is 500 milliseconds, so the first image capture device 122 captures every 100 milliseconds and the second image capture device 124 captures every 500 milliseconds; one second image 120 is captured for every five first images 118. The intermittent capturing by the second image capture device 124 may be accompanied by a coinciding intermittent flash of an illumination source. For example, if fluoresced light is to be captured by the second image capture device 124, then a corresponding light source may flash for enough duration to allow a single second image 120 to be captured by the second image capture device 124. With this technique, any effects of the fluorescing illuminant such as heat generation, interference with the first image capture device 122, etc., may be reduced. In some examples, the second images may be captured intermittently during capture of the first images such that some first images are captured for periods of time when no second images are captured.

In some embodiments, a single image sensor is used and only a single image (among all captured image sequences) is captured at any given time. The rate of capturing the second image sequence 116 may be selected based on what is imperceptible to the human eye (i.e., to human vision). For example, in such an embodiment the first image sequence 114 may be missing 1 frame every 1/60th of a second since the sensor is used at this time to capture an image in the second sequence 116. This approach may be imperceivable to a human when displayed as 60 frames per second. The sample rates discussed above are examples; the rate of sampling that may be imperceptible may depend on the display rate as well as other factors such as lighting conditions, characteristics of the scene, whether the image capture device is moving relative to the scene, etc.

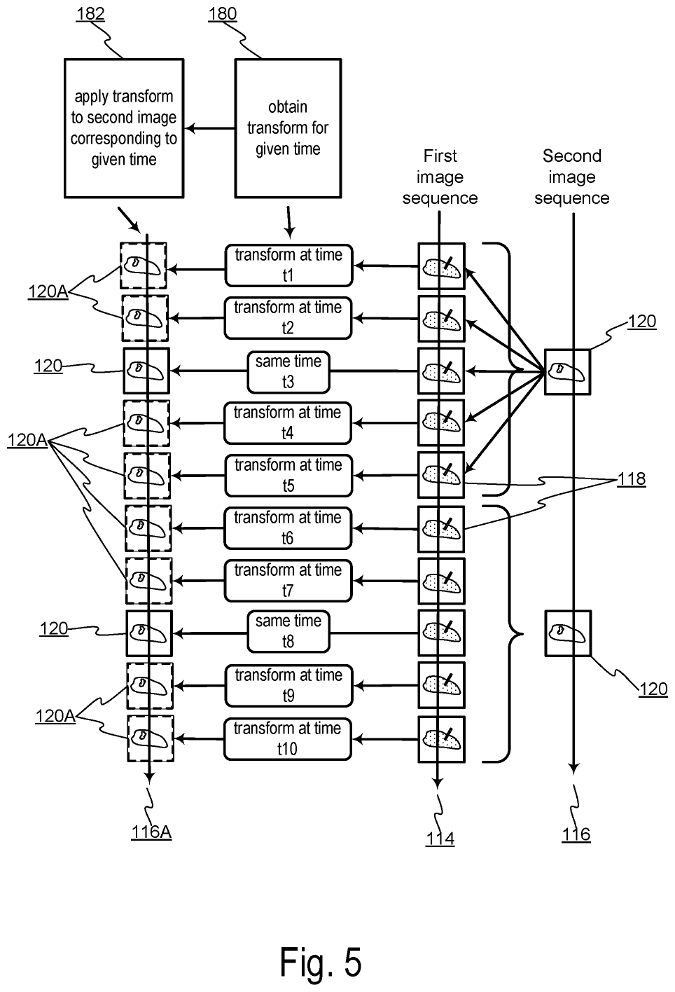

FIG. 5 shows a technique for generating synthetic second images 120A. As discussed above, the first image sequences 114 may have a higher frame rate than the second image sequence 116. Consequently, there may be first images 118 for which there are no corresponding (in time) second images 120. It may be helpful to “fill out” the gaps in the second image sequence 116 where there are no second images 120 that correspond (in time) to first images 118. As described below, some operations 154 with respect to the first image sequence 114 may be more feasible or accurate if each first image 118 has a corresponding second image 120.

One approach to filling out the second image sequence 116 (to match the first image sequence 114 image-by-image) is to duplicate second images 120. That is, for any given first image 118 for which no second image 120 was correspondingly captured, a closest (in time) captured second image 120. In other words, the second image sequence 116 may be filled to match the first image sequence 114 by filling the gaps in the first image sequence 114 with copies of closest (in time) second images 120. While this approach is efficient, if a first image 118 is captured at a given time and its corresponding second image 120 was captured at a significantly different time (e.g., several or many frames away) then then the pair of images may differ in ways that make later image processing less accurate. For example, if the first and second image capture devices 122, 124 are part of a same body (e.g., a camera or endoscope) and the body is moving while the first and second images 118,120 are being captured, then features in the copied second image 120 may not align with features in the first image 118. Moreover, light and surgical-scene conditions may change during the time between when the respective images were captured. Techniques may be used to generate synthetic second images 120A that may approximate second images that would have been taken by the second image capture device 124 at times when the first images 118 were captured.

One such technique is to, for any given first image 118 that has no time-corresponding second image 120, generate a corresponding transform. The transform is applied to the time-corresponding second image 120 to generate a synthetic second image 120A. The synthetic second image 120A may approximate what the second image capture device 124 would have captured if it had captured an image when the given first image 118 was captured. The transform may be a geometric transform. For example, a geometric transform may be generated based on information about the pose of a body or housing (e.g., a camera) that incorporates the first and second image capture devices 122, 124. The pose may be directly tracked by a separate system. Or the pose may be estimated by constructing a map of the scene from the captured images. Other techniques may be used. For example, a transform may be a linear interpolation between two second images 120. Color transforms may also be generated using interpolation. Regardless of how the synthetic second images 120A are generated, the result is that for each original captured second image 120 there will a number of corresponding synthetic second images 120A.

Referring to the example shown in FIG. 5, for each time shown (t1, . . . , t10), a transform is obtained 180 for the corresponding first image 118 and the transform is applied 182 to the corresponding second image 120 to generate a synthetic second image 120A for the time. For times when a first image 118 has a time-corresponding second image 120 (e.g., times t3 and t8), the transform may be an identity operation, or the transform may be omitted and the time-corresponding second image 120 is copied (or used). The resulting synthetic second image sequence 116A may be well-suited for the operations 154 described herein. As used herein, unless the context indicates otherwise, “second image sequence” refers to both the synthetic and the non-synthetic varieties.

While generating synthetic second images 120A is a useful technique, in other embodiments object recognition/detection may be performed on the original second images 120 rather than on a synthetic image sequence. The objects that are detected/recognized may propagated to subsequences of the first images 118. For example, referring to FIG. 5, an object detected/recognized in the second image 120 at time t3 may be propagated to the first images 118 at times t1 to t5. This technique may be used with any of the embodiments described herein that involve performing an operation with respect to the first image sequence 114 based on objects found in the second image sequence 116.

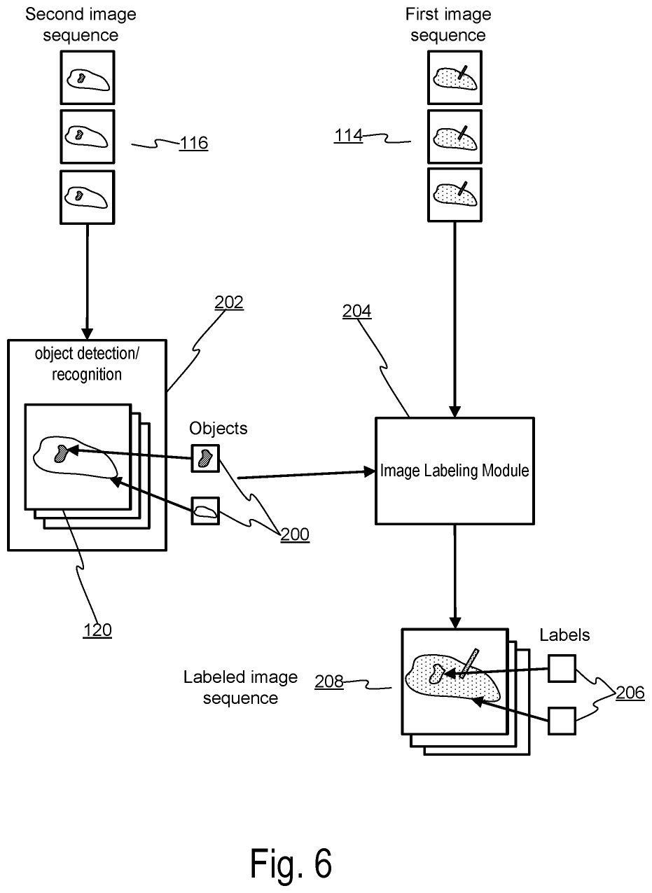

FIG. 6 shows an embodiment for labeling one or more first images 118 in a first image sequence 114 based on detecting and/or recognizing objects 200 based on one or more second images 120 in a second image sequence 114. In this embodiment, the second image sequence 116 is passed through an object detection/recognition module 202. The object detection/recognition module 202 may implement any algorithms for detecting objects and/or recognizing objects in images. Temporal algorithms that use inter-frame analysis may be used to detect and/or recognize objects 200. Indications of the detected/recognized objects 200 are passed to an image labeling module 204 (described below). In the case of object detection, the indications may include information about the extent and location of objects detected in the second images 120. In some embodiments, the indications may include bitmasks representing detected objects. In the case of object recognition, the indications may also (or alternatively) include information about the types or categories of objects recognized in the second images 120. In sum, the object detection/recognition module 202 may output indications of detected/recognized objects 200 to the image labeling module 204.

In some embodiments, the second image sequence 116 may be input into the object detection/recognition module 202 instead of the first image sequence 114 due to differences in the imaging modalities of the first image sequence 114 and the second image sequence 116. In such embodiments, performing object detection/recognition using the second image sequence 116 may produce more accurate labels and/or require less computational resources than performing a similar object detection/recognition using the first image sequence 114. Consider, for example, an embodiment in which the first image sequence 114 includes visible light images and the second image sequence 116 includes fluorescence images. In such an embodiment, areas of fluorescence signal in an image in the second image sequence 116 may correspond to certain objects such as a perfused organ. The areas of fluorescence signal may therefore be used to detect and/or recognize the organ in the image without relying on more computationally expensive operations that may be required to detect and/or recognize the same organ in a corresponding visible light image.

The image labeling module 204 may use the indications of detected/recognized objects 200 to perform operations with respect to the first image sequence 114. The image labeling module 204 also receive the first image sequence 114 and synchronizes the indications with the individual first images 118. In some embodiments, the synchronization may be inherent, for example the image labeling module 204 may be driven by a common timer or clock (as part of an image processing pipeline). In other embodiments, the synchronization may be based on timestamps included with the indications; the timestamps may be used to align the respective detected/recognized objects 200 with individual first images 118.

The image labeling module 204 may use the indications to label the first images 118. If the indications indicate recognized objects or categories, then the first images 118 may be labeled accordingly. A label 206 may be added to metadata of a first image 118 or may be kept in a separate data structure linked to the first image 118. If the indications include locations (and/or shapes) of objects, then the locations may be included in the metadata or data structure. In sum, the objects detected/recognized in the second image sequence 116 may be synchronously associated with the first image sequence 114. The resulting labeled image sequence 208 may be used for other purposes, either alone or in combination with the second image sequence 116. Alone, one or more labeled images in the labeled image sequence 208 can be used for supervised machine learning, for example. A computer-assisted medical system may also make use of the labels 206 to make decisions, control movements, render sound or graphics, generate a model of the scene 102, etc.

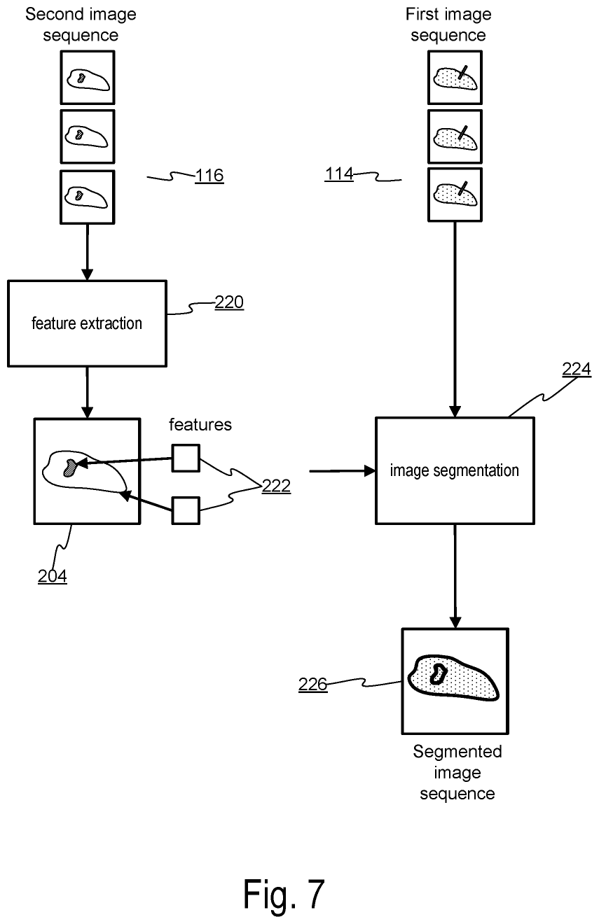

FIG. 7 shows an embodiment for segmenting a first image sequence 114. A feature extraction module 220 receives a second image sequence 116 and performs any known feature extraction algorithm to extract features 222 from the second image sequence 116. In one embodiment, the feature extraction algorithm may be the object detection/recognition module 202 and the features 222 are detected/recognized objects. In other embodiments, the feature extraction algorithm may extract other types of features from the second image sequence 116. For example, the feature detection algorithm may extract features 222 such as one or more of: edges, segments (e.g., using background-foreground segmentation), a color histogram (or other statistics about the second images 120), gradient maps, blobs, key points or points of interest, or other known image features.

The features 222 are passed to an image segmentation module 224, which also receives the first image sequence 114. As with the object detection/recognition embodiment described above, indications of the extracted features 222 may be synchronized to the first images 118. In one embodiment, the image segmentation module may directly apply segments from the second image sequence 116, i.e., segment the first images 118 in the same way their respective counterpart second images 120 are segmented. In another embodiment, the features 222 may provide additional information for segmenting the first images 118. In yet another embodiment, the segmentation module 224 may segment the first images 118 and then join those segments with segments from the second images 120. The segmentation module 224 outputs a segmented image sequence 226, which may be used for further image processing, as input to a computer-assisted medical system, etc.

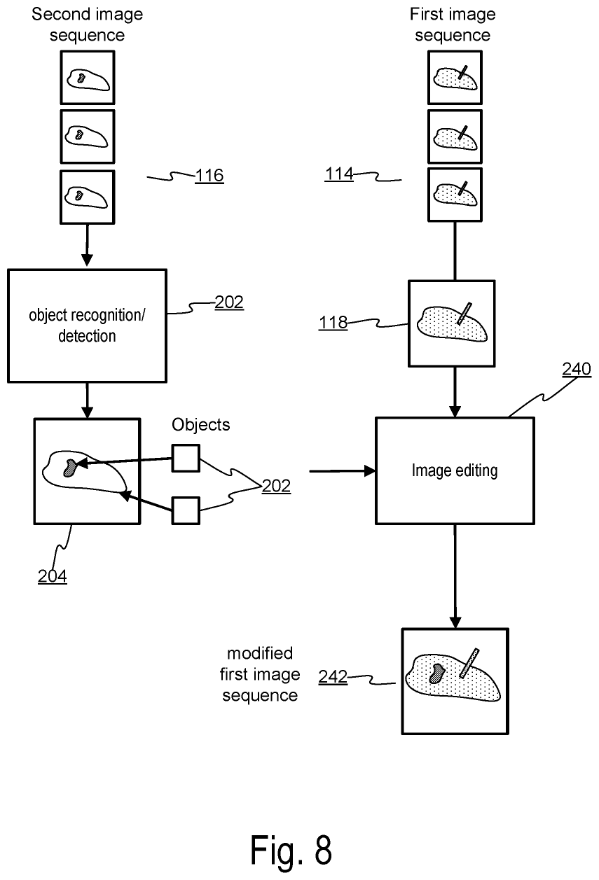

FIG. 8 shows an embodiment for modifying a first image sequence 114. As discussed above with reference to FIG. 6, object detection/recognition may be performed on the second image sequence 116. Indications thereof may be provided to an image editing module 240, as well as the first image sequence 114. The indications of detected/recognized objects may be synchronized to the second image sequence 116. Any given first image 118 may be modified by the image editing module 240 based on whichever detected/recognized objects are chronologically associated with the given first image 118. For example, detected/recognized objects may be merged with the first images 118. In another embodiment, objects in the first images 118 may be correlated with objects detected in the second images 120, which may be colored, highlighted, etc. according to the objects in the first images 118. In another embodiment, objects recognized in the second images 120 may be correlated with objects in the first images 118, e.g., by comparing locations, shape similarity, etc. The objects in the first images 118 are then modified according to the labels of the objects recognized in the second images 120. For example, if an object is recognized as a particular anatomical feature such as an artery, then a graphic “artery” label may be added to the corresponding first images 118. As another example, a shape in a first image 118 that has been correlated with an object recognized in a second image 120 may be colored according to the label of the recognized object. As another example, a graphical element representative of the recognized object may be added (overlaid) in a first image 118 but be colored according to color in the overlay location of the first image 118 (before overlaying the recognized object). The image editing module 240 may graphically alter the first image sequence 114 in any way that makes use of the objects recognized/detected in the second image sequence 116, thus producing a modified first image sequence 242.

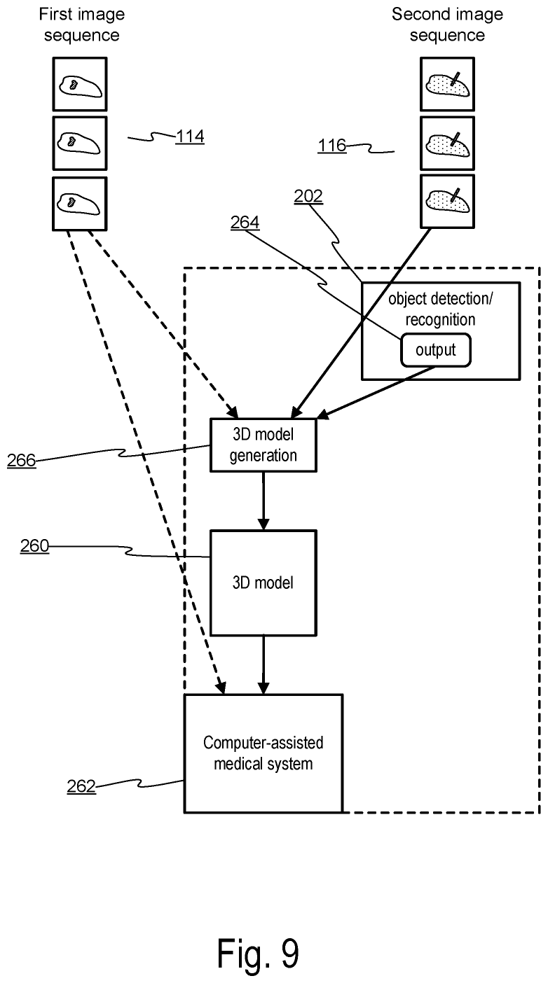

FIG. 9 shows an embodiment for reconstructing at least portions of a scene as a 3D model 260 and using the 3D model 260. While this embodiment may be useful when implemented by a computer-assisted medical system 262, any image processing system 126 may be used. As discussed above, an object detection/recognition module 202 detects and/or recognizes objects in the second image sequence 116 and provides outputs 264 about detected/recognized objects. A 3D model generation module 266 receives the outputs 264 and may also receive the first and/or second image sequences 114,116. The 3D model generation module 266 may generate the 3D model 260 from the image sequences using known model generation algorithms, possibly making use of information about poses of the image capture devices when images in the image sequences were captured.

In one embodiment, a 3D model may be generated from each respective image sequence and the models may be merged to form the 3D model 260. In another embodiment, 3D model data from the second image sequence 116 may be used to generate a 3D model of internal (sub-surface) structure of tissue, organ, or other anatomical features that may not be visible in the first image sequence 114. Any of the 3D models mentioned may be rendered and displayed by the computer-assisted medical system 262. In some examples, displaying the 3D model may include overlaying a perspective-aligned view of the 3D model over the first image sequence 114 or otherwise compositing the perspective aligned view of the 3D model with the first image sequence 114 to produce an augmented view of the scene. Any of the models may be supplemented with the objects detected/recognized in the second image sequence 116. For example, if an object is recognized as an anatomical feature, that feature might be added to a model as linked metadata or a graphic label (to be rendered). A detected/recognized object may indicate which textures to apply to a 3D model, etc. In some embodiments, movement of a component of the computer-assisted medical system 262 may be controlled based on one or more of the 3D models. For example, the 3D model 260 might include a representation of a critical anatomical feature (e.g., a nerve) and the computer-assisted medical system 262 may inhibit a movable element thereof from contacting (or approaching) the critical anatomical feature. This may be done, e.g., by using the generated 3D model to generate a no-fly zone at a location corresponding to the physical object represented by the 3D model. A movable element (e.g., a distal end of an instrument mounted to a manipulator arm) may be controlled to be prevented from entering the no-fly zone. The model generation process may also be used to estimate anatomical dimensions based on recognized objects.

In some embodiments, the object detection/recognition may involve techniques more specific to the type of content expected in the second image sequence 116. For example, the values of pixels in second images 120 may be indicative of types of molecules. Consequently, pixels, regions, blobs, objects, etc. may be recognized as corresponding to types of molecules and may be tagged accordingly. In addition, tags of anatomical features recognized in the second image sequence 116 may be used by other algorithms such as an algorithm for identifying stages of surgical procedures, an algorithm for identifying a type of medical procedure, or an algorithm for evaluating the accuracy of molecule tagging.

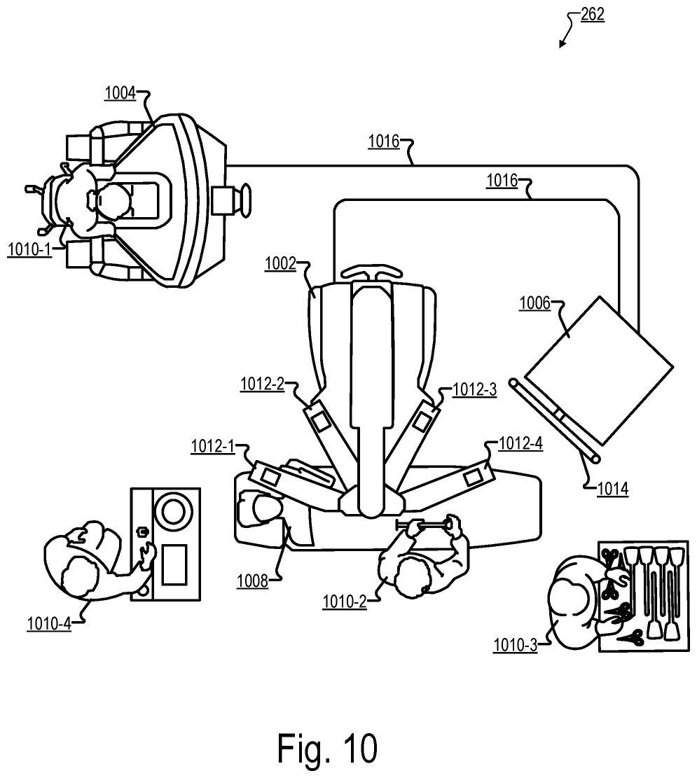

As has been described, the imaging device 112 and/or image processing system 126 may be associated in certain examples with a computer-assisted medical system used to perform a medical procedure on a body (whether alive or not). To illustrate, FIG. 10 shows an example of a computer-assisted medical system 262 that may be used to perform various types of medical procedures including surgical and/or non-medical procedures. The imaging device 112 and the image processing system 126 may be part of, or supplement, the computer-assisted medical system 262. The computer-assisted medical system 262 may make use of the image sequences as they are captured in real-time during a surgical procedure. For example, by triggering recording of video (image sequences), updating a user interface, rendering (audio/video) a notification during a surgical procedure, etc.

As shown, the computer-assisted medical system 262 may include a manipulator assembly 1002 (a manipulator cart is shown in FIG. 10), a user control apparatus 1004, and an auxiliary apparatus 1006, all of which are communicatively coupled to each other. The computer-assisted medical system 262 may be utilized by a medical team to perform a computer-assisted medical procedure or other similar operation on a body of a patient 1008 or on any other body as may serve a particular implementation. As shown, the medical team may include a first user 1010-1 (such as a surgeon for a medical procedure), a second user 1010-2 (such as a patient-side assistant), a third user 1010-3 (such as another assistant, a nurse, a trainee, etc.), and a fourth user 1010-4 (such as an anesthesiologist for a medical procedure), all of whom may be collectively referred to as users 1010, and each of whom may control, interact with, or otherwise be a user of the computer-assisted medical system 262. More, fewer, or alternative users may be present during a medical procedure as may serve a particular implementation. For example, team composition for different medical procedures, or for non-medical procedures, may differ and include users with different roles.

Some embodiments may be implemented in the context of components of the computer-assisted medical system 262. For example, the image capture device 122 may be part of an endoscope mounted to one of the manipulator arms 1012 or may held by a user (e.g., user 1010-2). Any one or more of the first image sequence 114, second images sequence 116, or results of operations 154/154A performed with respect to the first image sequence 114 may be displayed using a display in the user control apparatus 1004 and/or using a display monitor 1014. The image processing system 126 may be implemented using computer systems at any of an endoscope, the auxiliary apparatus 1006, the control apparatus 1004 or other computing systems not shown in FIG. 10.

While FIG. 10 illustrates an ongoing minimally invasive medical procedure such as a minimally invasive medical procedure, it will be understood that the computer-assisted medical system 262 may similarly be used to perform open medical procedures or other types of operations. For example, operations such as exploratory imaging operations, mock medical procedures used for training purposes, and/or other operations may also be performed.

As shown in FIG. 10, the manipulator assembly 1002 may include one or more manipulator arms 1012 (e.g., manipulator arms 1012-1 through 1012-4) to which one or more instruments may be coupled. The instruments may be used for a computer-assisted medical procedure on patient the 1008 (e.g., in a surgical example, by being at least partially inserted into the patient 1008 and manipulated within the patient 1008). While the manipulator assembly 1002 is depicted and described herein as including four manipulator arms 1012, the manipulator assembly 1002 may include a single manipulator arm 1012 or any other number of manipulator arms. While the example of FIG. 10 illustrates the manipulator arms 1012 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to the manipulator arms 1012 shown in FIG. 10.

During the medical operation, the user control apparatus 1004 may be configured to facilitate teleoperational control by the user 1010-1 of the manipulator arms 1012 and instruments attached to the manipulator arms 1012. To this end, the user control apparatus 1004 may provide the user 1010-1 with imagery of an operational area associated with patient 1008 as captured by an imaging device. To facilitate control of instruments, user control apparatus 1004 may include a set of master controls. These master controls may be manipulated by the user 1010-1 to control movement of the manipulator arms 1012 or any instruments coupled to the manipulator arms 1012.

The auxiliary apparatus 1006 may include one or more computing devices configured to perform auxiliary functions in support of the medical procedure, such as providing insufflation, electrocautery energy, illumination or other energy for imaging devices, image processing, or coordinating components of computer-assisted medical system 262. In some examples, the auxiliary apparatus 1006 may be configured with a display monitor 1014 configured to display one or more user interfaces, or graphical or textual information in support of the medical procedure. In some instances, the display monitor 1014 may be implemented by a touchscreen display and provide user input functionality. Augmented content provided by a region-based augmentation system may be similar, or differ from, content associated with the display monitor 1014 or one or more display devices in the operation area (not shown).

The manipulator assembly 1002, user control apparatus 1004, and auxiliary apparatus 1006 may be communicatively coupled one to another in any suitable manner. For example, as shown in FIG. 10, the manipulator assembly 1002, user control apparatus 1004, and auxiliary apparatus 1006 may be communicatively coupled by control lines 1016, which may represent any wired or wireless communication link as may serve a particular implementation. To this end, the manipulator assembly 1002, user control apparatus 1004, and auxiliary apparatus 1006 may each include one or more wired or wireless communication interfaces, such as one or more local area network interfaces, Wi-Fi network interfaces, cellular interfaces, and so forth.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random-access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

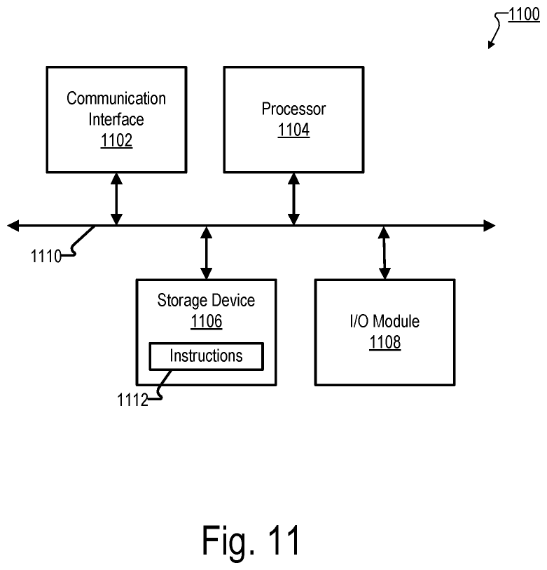

FIG. 11 shows an illustrative computing device 1100 that may be specifically configured to perform one or more of the processes described herein. Any of the systems, computing devices, and/or other components described herein may be implemented by the computing device 1100.

As shown in FIG. 11, the computing device 1100 may include a communication interface 1102, a processor 1104, a storage device 1106, and an input/output (“I/O”) module 1108 communicatively connected one to another via a communication infrastructure 1110. While an illustrative computing device 1100 is shown in FIG. 11, the components illustrated in FIG. 11 are not intended to be limiting. Additional or alternative components may be used in other embodiments. The computing device 1100 may be a virtual machine or may include virtualized components. Components of the computing device 1100 shown in FIG. 11 will now be described in additional detail.

The communication interface 1102 may be configured to communicate with one or more computing devices. Examples of the communication interface 1102 include, without limitation, a wired network interface (such as a network interface card), a wireless network interface (such as a wireless network interface card), a modem, an audio/video connection, and any other suitable interface.

The processor 1104 generally represents any type or form of processing unit capable of processing data and/or interpreting, executing, and/or directing execution of one or more of the instructions, processes, and/or operations described herein. The processor 1104 may perform operations by executing computer-executable instructions 1112 (e.g., an application, software, code, and/or other executable data instance) stored in the storage device 1106.

The storage device 1106 may include one or more data storage media, devices, or configurations and may employ any type, form, and combination of data storage media and/or device. For example, the storage device 1106 may include, but is not limited to, any combination of the non-volatile media and/or volatile media described herein. Electronic data, including data described herein, may be temporarily and/or permanently stored in the storage device 1106. For example, data representative of computer-executable instructions 1112 configured to direct the processor 1104 to perform any of the operations described herein may be stored within the storage device 1106. In some examples, data may be arranged in one or more databases residing within the storage device 1106.

The I/O module 1108 may include one or more I/O modules configured to receive user input and provide user output. The I/O module 1108 may include any hardware, firmware, software, or combination thereof supportive of input and output capabilities. For example, the I/O module 1108 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

The I/O module 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O module 1108 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
one or more processors communicatively coupled to the memory and configured to execute the instructions to perform a process comprising:
accessing a first image sequence comprising first images, the first images based on illumination, using visible-spectrum light, of a scene associated with a medical procedure;
accessing a second image sequence comprising second images, the second images based on illumination of the scene using non-visible spectrum light;
detecting, based on an inter-frame analysis of the second images, an object in the second image sequence; and
performing, based on the detected object, an operation with respect to the first image sequence.

2. The system according to claim 1, wherein the non-visible spectrum light comprises fluoresced light.

3. The system according to claim 1, wherein the non-visible spectrum light comprises infrared light.

4. The system according to claim 1, wherein the first images are captured at a first rate of images-per-second, wherein the second images are captured at a second rate of images per second, and wherein the second rate is less than the first rate.

5. The system according to claim 1, wherein the process further comprises accessing a third image sequence comprising third images, detecting a second object in the third image sequence, and performing the operation based on the detected object and the second detected object.

6. The system according to claim 1, wherein an image capture device captures the first image sequence and the second image sequence while the image capture device and the scene are moving relative to each other, wherein a first image in the first image sequence is captured at a first time and a second image in the second image sequence is captured at a second time, and wherein the second image is geometrically transformed based on data indicating movement of the image capture device between the first time and the second time.

7. The system according to claim 1, wherein the process further comprises recognizing the object detected in the second image sequence, and wherein the operation comprises applying a label to the first image sequence based on the recognizing of the object.

8. The system according to claim 1, wherein the operation comprises modifying pixels in a first image based on the object detected in the second image sequence.

9. The system according to claim 1, wherein the process further comprises accessing a third image sequence comprising third images, detecting a second object in the third image sequence, and performing a second operation with respect to the first image sequence based on the detected second object.

10. The system according to claim 1, wherein a subsequence of first images in the first image sequence chronologically corresponds to a second image in the second image sequence, wherein the process further comprises recognizing the object detected in the second image sequence, wherein the recognized object comprises a recognized anatomical feature, and wherein an indication of the recognized anatomical feature is applied to the subsequence of first images.

11. The system according to claim 10, wherein the applying comprises labeling or modifying the subsequence of first images.

12. The system according to claim 1, wherein light provided by an illuminant in correspondence with capturing the second image sequence is imperceptible to human vision.

13. The system according to claim 1, wherein the second images are captured intermittently during capture of the first images such that some first images are captured for periods of time when no second images are captured.

14. The system according to claim 13, wherein the first images and the second images are captured by a same sensor, and wherein the first images and the second images are captured at mutually exclusive times.

15. The system according to claim 1, wherein the process further comprises recognizing the object detected in the second image sequence, and wherein a label corresponding to the recognized object is displayed with the first image sequence.

16. The system according to claim 1, wherein a segmentation of a first image is displayed based on the detected object.

17. The system according to claim 1, wherein a computer-assisted medical system is controlled based on the detected object.

18. The system according to claim 17, wherein the computer-assisted medical system comprises a manipulator arm, and wherein the controlling comprises inhibiting movement of the manipulator arm based on the detected object.

19-29. (canceled)

30. A method performed by one or more computing devices, the method comprising:
accessing a first image sequence comprising first images, the first images based on illumination, using visible-spectrum light, of a scene associated with a medical procedure;
accessing a second image sequence comprising second images, the second images based on illumination of the scene using non-visible spectrum light;
detecting, based on an inter-frame analysis of the second images, an object in the second image sequence; and
performing, based on the detected object, an operation with respect to the first image sequence.

31-58. (canceled)

59. A non-transitory computer-readable medium storing instructions that, when executed, direct a processor of a computing device to perform a process comprising:
accessing a first image sequence comprising first images, the first images based on illumination, using visible-spectrum light, of a scene associated with a medical procedure;
accessing a second image sequence comprising second images, the second images based on illumination of the scene using non-visible spectrum light;
detecting, based on an inter-frame analysis of the second images, an object in the second image sequence; and
performing, based on the detected object, an operation with respect to the first image sequence.
